(19) United States
(12) Patent Application Publication
(10) Pub. No.: US 2004/0087539 A1
(43) Pub. Date: May 6, 2004

(54) Method of treating conditions related to platelet activity

(76) Inventor: Xiaoping Du, Westmont, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/467,387

(22) PCT Filed: Feb. 5, 2002
(86) PCT No.: PCT/US02/03372

(51) Int. Cl. A61K 31/522

(52) U.S. Cl. 514/45; 514/263.3

(57) ABSTRACT

Methods of treating thrombotic and hemostatic conditions related to platelet activity are described herein. The methods of treating thrombotic and hemostatic conditions use active agents that modulate production of guanosine 3′, 5′ cyclic monophosphate (cGMP) or the function of cGMP-dependent protein kinase (PKG), and its downstream effectors, the ERK and p38 pathways.

### CROSS REFERENCE TO RELATED APPLICATION

This application claims the benefit of U.S. Provisional Patent Application No. 60/267,326, filed Feb. 8, 2001.

### STATEMENT OF GOVERNMENTAL INTEREST

The subject matter of this application has been supported in part by research grant Grant Nos. HL 52547 and HL 62350 from the National Institute of Health (NIH), Bethesda, Md., U.S.A.

### FIELD OF THE INVENTION

The present invention relates to methods of treating thrombotic and hemostatic conditions related to platelet activity enhanced by the guanosine 3′,5′-cyclic monophosphate (cGMP)-dependent protein kinase signaling pathway. One aspect of the present invention relates to a method of treating a thrombotic condition by using an active agent that inhibits production of guanosine 3′,5′-cyclic monophosphate or that inhibits cGMP-dependent protein kinase (protein kinase G, PKG) in platelets. Another aspect of the present invention relates to a method of treating a hemostatic condition. The hemostatic condition can be treated by administering an active agent that stimulates production of cGMP or that stimulates the function of PKG in platelets.

### BACKGROUND OF THE INVENTION

The role of integrins in cell-adhesion, among other pathways, is well-known. It is commonly understood that integrins are heterodimers that actively participate in platelet activity. The heterodimers generally have an α and β subunit. The prototype integrin, α_{IIb}β₃, on nonactivated platelets circulating in the bloodstream has a low affinity for binding to its ligands, for example fibrinogen. At sites of vascular injury, exposure of the platelets to various soluble agonists, such as thrombin and adenosine diphosphate (ADP), or subendoethelial adhesive proteins, for example collagen and von Willebrand factor (vWF), induces a series of morphological and biochemical changes, i.e., platelet activation. Platelet activation leads to enhanced ligand binding function of integrin α_{IIb}β₃ and, accordingly, adhesion and aggregation of the platelets.

Under high shear rate conditions, such as in stenotic arteries, the initial platelet adhesion is dependent on the platelet vWF receptor, i.e., the glycoprotein Ib-IX complex (GPIb-IX), which induces signal activating ligand binding function of integrin α_{IIb}β₃. The important role of integrins in platelet adhesion and aggregation has been linked causally to thrombotic diseases, for example, heart attack, stroke, and hemostasis.

The function of PKG is stimulated by cGMP. It has been reported that platelet aggregation can be inhibited by nitric oxide (NO) and nitroprusside, agents that elevate intracellular cGMP and adenosine 3′,5′-cyclic monophosphate (cAMP). It has been theorized that elevated levels of cGMP stimulate cGMP-dependent protein kinase production, which inhibits platelet activity.

One active agent, sildenafil, is commercially available from Pfizer, Inc. (New York, N.Y., U.S.A.) as VIAGRA®. Sildenafil enhances intracellular levels of cGMP by inhibiting phosphodiesterase 5 (PDE5), an enzyme that hydrolyzes cGMP. Under the currently accepted theory that cGMP and PKG inhibit platelet activity, cGMP-enhancing drugs, such as sildenafil, are expected to inhibit platelet activation and thereby reduce incidences of thrombosis. However, it has been found that sildenafil does not inhibit platelet activation. In addition, an increasing number of clinical reports associate severe thrombotic conditions with the use of sildenafil, which indicates that, unlike nitric oxide and nitroprusside, specific cGMP-enhancing drugs do not inhibit platelet activation.

Accordingly, it would be beneficial to better understand the role of cGMP and PKG in platelet activation. The identification of key components in the cGMP-PKG pathway and their function would provide new methods for modulating integrin-dependent platelet aggregation. A more thorough understanding of the cGMP-PKG pathway in platelets could lead to new and improved methods and active agents for treating thrombotic conditions as well as hemostatic conditions, and provide potential mechanisms for identifying agents useful in reducing or eliminating thrombotic complications associated with cGMP-enhancing agents, such as sildenafil.

cGMP-dependent protein kinase is a family of intracellular signaling enzymes widely distributed in various tissues and cells. PKG signaling plays a significant role in the cGMP-PKG pathway, which mediates platelet activation. It is theorized, but not relied upon herein, that PKG catalyzes phosphorylation of several cytoskeletal and signaling molecules useful in cytoskeleton organization, cell migration, secretion and neuron signal transmission, including vasodilator-stimulated phosphoprotein, upon activation by cGMP binding.

The present investigation indicates that cGMP induces biphasic platelet response. In the early phase, cGMP enhances activation of integrin α_{IIb}β₃ and integrin-dependent platelet aggregation induced by vWF and thrombin. It also was found that binding of vWF to the platelet vWF receptor induces and enhances cGMP level and activate platelets via the PKG-cRaf-MEK (mitogen-activated protein kinase/-extracellular signal-regulated kinase kinase)-ERK (extracellular signal-regulated kinase) pathway. Furthermore, the p38 mitogen-activated protein kinase (MAPK) pathway also is important in mediating PKG-dependent activation of platelets. See Z. Li et al., J. Biol. Chem., 276(45), pp. 42226-42232 (Nov. 9, 2001).

In light of this understanding of platelet activation, and the stimulatory roles of PKG in platelet activation, new methods and new uses of active agents are disclosed for treating thrombotic conditions, as well as hemostatic conditions. Therefore, in one embodiment of the present invention, an inhibitor of guanylyl cyclase (an enzyme useful in cGMP production), an inhibitor of PKG, an activator of cGMP-specific phosphodiesterase, an enzyme that hydrolyzes cGMP, an inhibitor of the cRaf-MEK-ERK pathway, an inhibitor of the p38 pathway, or mixtures thereof, are used to treat thrombotic conditions, such as myocardial infarction, cerebral thrombosis, and other arterial thrombosis. In another embodiment of the present invention, activators of guanylyl cyclase, activators of PKG, and inhibitors of cGMP-specific phosphodiesterases (for example, PDE5), are administered to a patient to enhance platelet activities for treating hemostatic conditions, such as von Willebrand disease and thrombocytopenia.

Von Willebrand disease (vWD) can be caused by a decrease in the quantity of vWF (type I) or a decrease in the function of vWF (type IIA) to bind to its platelet receptor, GPIb-IX. It has been found that GPIb-IX induces platelet activation via a PKG-dependent pathway, enhancing PKG function or cGMP levels, which can be used to correct platelet dysfunction caused by a decrease in the quantity or function of vWF. Also, enhanced platelet activation via the PKG pathway can compensate for a decrease in platelet numbers or function in patients with thrombocytopenia or functional platelet disorders.

### SUMMARY OF THE INVENTION

In one aspect, the present invention provides a method of treating a thrombotic disease or condition, characterized by the formation, presence, or development of a blood clot in blood vessels. The method comprises the step of administering a therapeutically effective amount of an active agent that, directly or indirectly, inhibits production of guanosine 3′,5′-cyclic monophosphate (cGMP) or inhibits the function of PKG and its downstream effectors, cRaf-MEK-ERK pathways, to a mammal in need of such treatment. The active agent is selected from the group consisting of guanylyl cyclase inhibitors, cCMP-dependent phosphodiesterase activators, protein kinase G (cGMP-dependent protein kinase, PKG) inhibitors, and inhibitors of cRAF, MEK1, and ERK1/ERK2.

In another aspect, the present invention also provides a method of treating a hemostatic disease or condition. Hemostatic diseases and conditions are characterized by a decreased capacity of a human or animal to control or stop bleeding. The method of treating a hemostatic disease or condition comprises the step of administering a therapeutically effective amount of an active agent that stimulates production of guanosine 3′,5′-cyclic monophosphate or PKG to a mammal in need of such treatment. The active agent can be any compound that stimulates production of cGMP, inhibits cGMP-specific phosphodiesterase (e.g., phosphodiesterase 5), or stimulates activity of the PKG-cRaf-MEK-ERF pathway. Suitable active agents include, but are not limited to, compounds selected from the group consisting of guanylyl cyclase activators, protein kinase G activators, cGMP-specific phosphodiesterase inhibitors, and stimulators of cRaf, MEK, and ERK1/ERK2.

These and other aspects, advantages, and features of the invention will become apparent from the following detailed description of the preferred embodiments.

### BRIEF DESCRIPTION OF THE DRAWINGS

FIG. 1A shows effects on CHO cells transfected with cDNA encoding PKGIα or vector;

FIG. 1B contains bar graphs showing PKG activity in PKG-transfected cells;

FIG. 1C contains plots showing reconstitution of GPIb-IX-mediated activation in CHO cells;

FIGS. 2A-C contain bar graphs of tests performed on CHO cells for adhesion under flow;

FIGS. 3A-C contain plots and stains showing the effects of PKG inhibitors and activators on platelet aggregation induced by vWF-GPIb-IX interaction;

FIG. 4 contains bar graphs for cGMP concentration vs. a control and various test reactants;

FIGS. 5A and B are plots showing the effects of PKG inhibitors (A) and activators (B) on thrombin-induced platelet aggregation;

FIGS. 6A and B are plots showing the effects of sildenafil on platelet aggregation in the presence of ristocetin (A) and thrombin (B);

FIGS. 7A-D are plots showing the time-dependent biphasic effects of cGMP on GPIb-IX-dependent platelet aggregation; and

FIGS. 8A and B illustrate pathways for cGMP signaling in platelet activation and hemostasis.

### DETAILED DESCRIPTION OF THE PREFERRED EMBODIMENTS

The platelet integrin α_{IIb}β₃ mediates platelet adhesion and aggregation, and plays critical roles in the development of thrombotic diseases, such as heart attack and stroke. On circulating resting platelets, the integrin α_{IIb}β₃ has a low affinity for its ligands. At sites of vascular injury, various soluble platelet agonists (e.g., thrombin and ADP) or subendothelial adhesive proteins (e.g., collagen and von Willebrand factor (vWF)) induce platelet activation which represents a series of morphological and biochemical changes and intracellular signaling events, and is characterized by the activation of ligand binding function of integrin α_{IIb}β₃ (see Refs. 1-3).

Under moderate to high shear rate flow conditions, such as in stenotic artherosclerotic arteries, platelet adhesion and activation requires the platelet vWF receptor, i.e., glycoprotein Ib-IX complex (GPIb-IX). GPIb-IX interaction with the subendothelium-bound vWF initiates platelet adhesion and triggers integrin α_{IIb}β₃ activation, leading to integrin-dependent stable platelet adhesion and aggregation (see Refs. 4-14). Furthermore, GPIbIX binds thrombin, and is required for platelet aggregation induced by low-dose thrombin (see Refs. 15-21). The importance of GPIb-IX pathway in thrombosis and hemostasis is manifested in an inherited disease, i.e., Bernard-Soulier syndrome, in which genetic deficiency in GPIb-IX causes defects in platelet adhesion and activation, and results in bleeding disorder (see Ref. 22). The signaling pathways of GPIb-IX-mediated integrin activation are not totally clear. It has been found that the extracellular stimuli-responsive kinase (ERK) pathway mediates GPIb-IX-dependent integrin activation, and that cGMP-dependent protein kinase (protein kinase G, PKG) are important in promoting activation of ERK (see Ref. 23). However, these results apparently contradict the currently accepted theory that the cGMP-PKG pathway inhibits platelet activation.

cGMP is an important intracellular secondary messenger synthesized by guanylyl cyclases. Elevation of intracellular cGMP activates PKG, which catalyzes phosphorylation of several intracellular molecules, and regulates important cellular functions, such as cytoskeleton organization, cell migration, secretion, and neuron signal transmission (see Refs. 24-28). One important finding in PKG signaling is that the cGMP-PKG pathway mediates nitric oxide (NO)-induced vascular smooth muscle relaxation (see Refs. 29 and 30). Based on this mechanism, a specific cGMP-enhancing drug, sildenafil (VIAGRA®), has been developed and used to treat erectile dysfunction. Sildenafil selectively inhibits phosphodiesterase 5 (PDE5) that hydrolyzes cGMP, and thus enhances intracellular cGMP levels (see Ref. 31). The role of cGMP-PKG pathway in platelet activation was controversial in the early literature because increases in platelet cGMP levels had been observed in response to either platelet agonists (thrombin, ADP, or collagen) or inhibitors (NO donors such as sodium nitroprusside). The early theory that cGMP may play a role in promoting platelet activation was abandoned due to findings that preincubation of platelets with cGMP analogs or cGMP-enhancing agent NO inhibits platelet activation, and it has since been accepted by most investigators that cGMP inhibits platelet activation.

However, the controversy has not been resolved because early observations that cGMP is elevated by platelet agonists and that cGMP promotes platelet aggregation have not been explained (see Ref. 32-34). Also, the inhibitory effects of NO donors are complicated by findings that NO donors activate protein kinase A (PKA) pathway, which is known to potently inhibit platelet activation, and may directly affect the function of the integrin α_{IIb}β₃ (see Refs. 35-37). Furthermore, it has been shown that the sildenafil alone does not inhibit platelet activation in vitro, and there have been several clinical reports of severe thrombotic conditions (e.g., heart attack) associated with the use of sildenafil (see Refs. 38-42).

The thrombotic complication of sildenafil in some patients cannot be explained satisfactorily by the vasodilating effect of sildenafil because vasodilating drugs are used to treat angina pectoris. To better understand the roles of cGMP-PKG pathway in platelet activation and the underlying mechanisms of these apparent controversies, the roles of cGMP-PKG pathway in GPIb-IX-dependent platelet activation were investigated using the combination of molecular biological and pharmacological approaches. It was shown that elevation of cGMP following agonist stimulation promotes platelet activation induced by vWF or low dose thrombin. In contrast, cGMP inhibits subsequent platelet aggregation, when preincubated with platelets. Thus, cGMP has biphasic effects on platelets, i.e., an early phase transient stimulatory effect that mediates platelet activation and thus thrombus formation, and a second phase inhibitory effect that desensitizes platelets and serves to limit the size of thrombus.

For the purposes of the description herein, the term “treatment” includes preventing, lowering, stopping, or reversing the progression or severity of the condition or symptoms being treated. As such, the term “treatment” includes both medical therapeutic and/or prophylactic administration, as appropriate.

The methods of the present invention relate to diseases or conditions related to platelet activity. In one aspect, the condition is a thrombotic condition characterized by the formation, presence, or development of a thrombus, or blood clot. A thrombotic condition can include abnormal activation of blood clotting factors, for example increased platelet activation. Thrombotic conditions include, for example, myocardial infarction, cerebral thrombosis, arterial thrombosis, and occlusion of blood vessels.

Active agents suitable for treating thrombotic conditions are compounds that inhibit production of cGMP. Examples of suitable active agents for treating thrombotic conditions are, for example, compounds that (1) inhibit production of guanosine 3′,5′-cyclic monophosphate (cGMP) in platelets, including, but not limited to, a guanylyl cyclase inhibitor; (2) stimulate a cGMP-specific phosphodiesterase which hydrolyzes cGMP, including, but not limited to, a phosphodiesterase 5 activator; (3) inhibit activity of protein kinase G; (4) inhibit activity of a Raf, MEK, and ERKs, as well as the p38 pathway; and (5) mixtures of the above compounds. Compounds that can inhibit cGMP production include, but are not limited to, a guanylyl cyclase inhibitor, a protein kinase G inhibitor, and mixtures thereof. Examples of guanylyl cyclase inhibitors suitable for the invention include, but are not limited to, Ly83583, methylene blue, NS2028, ODQ, zinc (II) protoporphyrin, and mixtures thereof. Suitable protein kinase G inhibitors are KT5823, Rp-cGMP, Rp-8-bromo-cGMP, Rp-8-pCPT-cGMP, and mixtures thereof. Inhibitors of the cRaf-MEK-ERK pathway and p38 pathway include, but are not limited to, PD98059, U0125, U0126, ZM336372, and apigenin.

In particular, the following inhibitors can be used in the present method of treating thrombotic conditions:

cRaf inhibitors:

(N-[5-(dimethylaminobenzamide)-2-methylphenyl]-4-hydroxybenzamide (ZM336372);

MEK inhibitors:

2″-amino-3″-methoxyflavone (PD98059);

1,4-diamino-2,3-dicyano-1,4-bis(phenylthio)butadiene (U0125);

1,4-diamino-2,3-dicyano-1,4-bis (2-aminophenylthio)-butadiene (U0126);

2-(2-chloro-4-indo-phenylamino)-N-cyclopropylmethoxy-3,4-difluorobenzamide (PD184352);

ERK inhibitor:

4′,5,7-trihydroxyflavone (Apigenin);

p38 MAP kinase inhibitors:

4-(4-fluorophenyl)-2-(4-methylsulfinylphenyl)-5-(4-pyridyl)-1H-imidazole (SB203580);

4-(4-fluorophenyl)-2-(4-nitrophenyl)-5-(4-puridyl)-1H-imidazole (PD169316);

5-(2-amino-4-pyrimidinyl)-4-(4-fluorophenyl)-1-(4-piperidinyl)imidazole (SB220025);

2-methyl-4-phenyl-5-(4-pyridyl)oxazole (SC68376);

6-(4-fluorophenyl)-2,3-dihydro-5-(4-pyridyl)imidazo-[2,1-b]thiazole (SKF86002); and

4-(4-fluorophenyl)-2-(4-hydroxyphenyl)-5-(4-pyridyl)1H-imidazole (SB202190).

In another aspect of the present invention, hemostasis is characterized by controlling or stopping bleeding, for example in a surgical procedure or during hemorrhage. Hemostatic conditions can be related to a decreased ability of platelets to function properly, the lack of platelet activation, or a decrease in platelet count. Nonlimiting examples of hemostatic conditions include, but are not limited to, von Willebrand disease, thrombocytopenia, and the like.

Active agents suitable for treating hemostatic conditions are compounds that activate the production of cGMP. Examples of a suitable active agents useful in the method of treating hemostatic diseases and conditions include, but are not limited to, guanylyl cylcase activators, protein kinase G activators, and cGMP-specific phosphodiesterase inhibitors. A guanylyl cyclase activator suitable for the invention includes, but is not limited to, an atrial natriuretic peptide, a C-type natriuretic peptide, YC-1 (3-(5′-hydroxymethyl-2′-furyl)-1-benzylindazole), or a mixture thereof. A suitable protein kinase G activator includes, but is not limited, cGMP, 8-bromo-cGMP, 8-pCPT-cGMP, 8-dibutyl-cGMP, or a mixture thereof.

A cGMP-specific phosphodiesterase inhibitor suitable for use in the method is selected from the group consisting of sildenafil, E4021, DMPPO, MY5441, zaprinast, and the like. Additional cGMP-specific phosphodiesterase inhibitors are disclosed, for example, in International patent application WO96/16644, which is incorporated herein by reference. The active agents can include, but are not limited, the following, of which each patent or application is incorporated herein by reference;

i) a 5-substituted pyrazolo [4,3-d]-pyrimidine-7-one as disclosed in European patent application 02011889;

ii) a grisoleic acid derivative as disclosed in European patent applications nos. 0214708 and 0319050;

iii) a 2-phenylpurinone derivative as disclosed in European patent application 0293063;

iv) a phenylpyridione derivative as disclosed in European patent application 0347027;

v) a fused pyrimidine derivative as disclosed in European patent application 0347146;

vi) a condensed pyrimidine derivative as disclosed in European patent application 0349239;

vii) a pyrimidopyrimidine derivative as disclosed in European patent application 0351058;

viii) a purine compound as disclosed in European patent application 0352960;

ix) a quinazolinone derivative as disclosed in European patent application 0371731;

x) a phenylpyrimidone derivative as disclosed in European patent application 0395328;

xi) an imidazoquinoxalinone derivative or its aza analogue as disclosed in European patent application 0400583;

xii) a phenylpyrimidione derivative as disclosed in European patent application 0400799;

xiii) a phenylpyridione derivative as disclosed in European patent application 0428268;

xiv) a pyrimidopyrimadine derivative as disclosed in European patent application 0442204;

xv) a 4-aminoquinazoline derivative as disclosed in European patent application 0579496;

xvi) a 4,5-dihydro-4-oxo-pyrrolo[1,2-a]quinoxaline derivative or its aza analogue as disclosed in European patent application 0584487;

xvii) a polycyclic guanine derivative as disclosed in International patent application WO91/19717;

xviii) a nitrogenous heterocyclic compound as disclosed in International patent application WO93/07124;

xix) a 2-benzyl-polycyclic guanine derivative as disclosed in International patent application WO94/19351;

xx) a quinazoline derivative as disclosed in U.S. Pat. No. 4,060,615;

xxi) a 6-heterocyclyl oyrazolo [3,4-d]pyrimidin-4-one as disclosed in U.S. Pat. No. 5,294,612;

xxii) a benzimidazole as disclosed in Japanese patent application 5-222000; or

xxiii) a cycloheptimidazole as disclosed in European Journal of Pharmacology, 251, (1994), 1;

xxiv) a N-containing heterocycle as disclosed in International patent application WO94/22855;

xxv) a pyrazolopyrimidine derivative as disclosed in European patent application 0636626;

xxvi) a 4-aminopyrimidine derivative as disclosed in European patent application 0640599;

xxvii) an imidazoquinazoline derivative as disclosed in International patent application WO95/906648;

xxviii) an anthranilic acid derivative as disclosed in International patent application WO95/18097;

xxix) a 4-aminoquinazoline derivative as disclosed in U.S. Pat. No. 5,436,233;

xxx) a tetracyclic derivative as disclosed in U.S. Pat. No. 5,859,006;

xxxi) an imidazoquinazoline derivative as disclosed in European patent application 0668280; and

xxxii) a quinazoline compound as disclosed in European patent application 0669324.

The methods of the present invention include the use of any compound class disclosed in the patents and applications listed above as well as the particular individual compounds disclosed therein.

The methods of the present invention can be accomplished using an active agent as described above, or a physiologically acceptable salt or solvate thereof. The compound, salt, or solvate can be administered as the neat compound, or as a pharmaceutical composition containing either entity.

The active agents can be administered by any suitable route, for example by oral, buccal, inhalation, sublingual, rectal, vaginal, transurethral, nasal, topical, percutaneous, i.e., transdermal, or parenteral (including intravenous, intramuscular, subcutaneous, and intracoronary) administration. Parenteral administration can be accomplished using a needle and syringe, or using a high pressure technique, like POWDERJECT™.

The compounds and pharmaceutical formulations thereof include those wherein the active ingredient is administered in an effective amount to achieve its intended purpose. More specifically, a “therapeutically effective amount” means an amount effective to prevent development of, or to alleviate the existing symptoms of, the subject being treated. Determination of the effective amounts is well within the capability of those skilled in the art, especially in light of the detailed disclosure provided herein.

A “therapeutically effective dose” refers to that amount of the compound that results in achieving the desired effect. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. Compounds which exhibit high therapeutic indices are preferred. The data obtained from such data can be used in formulating a range of dosage for use in humans. The dosage of such compounds preferably lies within a range of circulating concentrations that include the ED₅₀ with little or no toxicity. The dosage can vary within this range depending upon the dosage form employed, and the route of administration utilized.

The exact formulation, route of administration, and dosage can be chosen by the individual physician in view of the patient's condition. Dosage amount and interval can be adjusted individually to provide plasma levels of the active moiety which are sufficient to maintain the therapeutic effects.

The amount of composition administered is dependent on the subject being treated, on the subject's weight, the severity of the affliction, the manner of administration, and the judgment of the prescribing physician.

Specifically, for administration to a human in the curative or prophylactic treatment of the conditions and disorders identified above, oral dosages of a compound of formula (I) generally are about 0.5 to about 1000 mg daily for an average adult patient (70 kg). Thus, for a typical adult patient, individual tablets or capsules contain 0.2 to 500 mg of active compound, in a suitable pharmaceutically acceptable vehicle or carrier, for administration in single or multiple doses, once or several times per day. Dosages for intravenous, buccal, or sublingual administration typically are 0.1 to 500 mg per single dose as required. In practice, the physician determines the actual dosing regimen which is most suitable for an individual patient, and the dosage varies with the age, weight, and response of the particular patient. The above dosages are exemplary of the average case, but there can be individual instances in which higher or lower dosages are merited, and such are within the scope of this invention.

For human use, a compound for the method of the invention can be administered alone, but generally is administered in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of compounds of formula (I) into preparations which can be used pharmaceutically.

These pharmaceutical compositions can be manufactured in a conventional manner, e.g., by conventional mixing, dissolving, granulating, dragee-making, emulsifying, encapsulating, entrapping, or lyophilizing processes. Proper formulation is dependent upon the route of administration chosen. When a therapeutically effective amount of a compound of the present invention is administered orally, the composition typically is in the form of a tablet, capsule, powder, solution, or elixir. When administered in tablet form, the composition can additionally contain a solid carrier, such as a gelatin or an adjuvant. The tablet, capsule, and powder contain about 5% to about 95% compound of the present invention, and preferably from about 25% to about 90% compound of the present invention. When administered in liquid form, a liquid carrier such as water, petroleum, or oils of animal or plant origin can be added. The liquid form of the composition can further contain physiological saline solution, dextrose or other saccharide solutions, or glycols. When administered in liquid form, the composition contains about 0.5% to about 90% by weight of a compound of the present invention, and preferably about 1% to about 50% of a compound of the present invention.

When a therapeutically effective amount of a compound of the present invention is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound of the present invention, an isotonic vehicle.

Suitable active agents can be readily combined with pharmaceutically acceptable carriers well-known in the art. Such carriers enable the present compounds to be formulated as tablets, pills, dragees, capsules, liquids, gels, syrups, slurries, suspensions and the like, for oral ingestion by a patient to be treated. Pharmaceutical preparations for oral use can be obtained by adding the active agent with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added.

For administration by inhalation, the active agents are conveniently delivered in the form of an aerosol spray presentation from pressurized packs or a nebulizer, with the use of a suitable propellant. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

The active agents can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical formulations for parenteral administration include aqueous solutions of the active agent in water-soluble form. Additionally, suspensions of the active agents can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

The active agents also can be formulated in rectal compositions, such as suppositories or retention enemas, e.g., containing conventional suppository bases. In addition to the formulations described previously, the compounds also can be formulated as a depot preparation. Such long-acting formulations can be administered by implantation (for example, subcutaneously or intramuscularly) or by intramuscular injection. Thus, for example, the active agents can be formulated with suitable polymeric or hydrophobic materials (for example, as an emulsion in an acceptable oil) or ion exchange resins, or as sparingly soluble derivatives, for example, as a sparingly soluble salt.

As previously mentioned, the active agents can be provided as salts with pharmaceutically compatible counterions. Such pharmaceutically acceptable base addition salts are those salts that retain the biological effectiveness and properties of the free acids, and that are obtained by reaction with suitable inorganic or organic bases.

In particular, an active agent can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A compound also can be injected parenterally, for example, intravenously, intramuscularly, subcutaneously, or intracoronarily. For parenteral administration, the compound is best used in the form of a sterile aqueous solution which can contain other substances, for example, salts, or monosaccharides, such as mannitol or glucose, to make the solution isotonic with blood.

For veterinary use, the active agent or a nontoxic salt thereof, is administered as a suitably acceptable formulation in accordance with normal veterinary practice. The veterinarian can readily determine the dosing regimen and route of administration that is most appropriate for a particular animal.

### Experimental Procedures

Effects of Recombinant PKG Expression on GPIb-JX-mediated Integrin Activation

Human PKG Iβ cDNA was cloned by RT-PCR, using human platelet mRNA as templates. Sequence of the human PKG Iβ cDNA fragment matches the published sequence. Cloning of human PKG Iα cDNA was described previously. PKG Iα or Iβ in pCDNA3.1/Zeo+vector was transfected into 123 cells (CHO cells expressing recombinant human CPIb-IX and integrin α_{IIb}β₃ using Lipofectamine plus (BRL). Expression of PKG was assessed by Western blotting with an antihuman PKG I antibody (Calbiochem). The kinase activity of the expressed enzymes was measured in cell homogenates using BPDEtide (Calbiochem) as substrate as detailed previously. vWF-induced GPIb-IX-mediated activation of integrin α_{IIb}β₃ was examined by flow cytometry analysis of Oregon green-labeled fibrinogen binding to integrln α_{IIb}β₃ as described previously. Briefly, cells were detached from tissue culture plate using 0.5 mM EDTA, washed at 4° C. and resuspended in modified Tyrode's solution. Cells then were incubated with Oregon green-labeled fibrinogen (20 μg/ml) in the presence of ristocetin and vWF at 22° C. for 30 minutes, and analyzed by FACScalibur flow cytometer. Nonspecific binding of fibrinogen was estimated by measuring fibrinogon binding in the presence of a specific integrin inhibitor RGDS (1 mM) (see Refs. 13 and 43-45).

Cell Adhesion to vWF Under Flow

CHO cells expressing human platelet receptors were resuspended in modified Tyrode's buffer, pH 7.4, containing 1 mg/mi of bovine serum albumin (BSA) (see Ref. 46), and final cell count was 5×10⁵/ml. Cell interaction with immobilized vWF under flow conditions was observed in real time under a Nikon inverted microscope equipped with a cooled CCD camera. Purified human vWF (10 μg/ml in 0.1 M NaHCO₃, pH 8.3) was added to glass microcapillary tubes (inner diameter of 1.33 mm and a length of 7.2 cm), and incubated at 4° C. for 20 h. The vWF-coated microcapillary tubes then were incubated with 5% BSA. After washing, the cell suspension was aspirated through the tube by a syringe pump (Harvard Apparatus Inc.) at desired shear rates for 1.2 minutes and washed with modified Tyrode's buffer for additional 10 minutes at the same shear rates to remove transiently adherent cells. The experiments were continuously recorded on videotape using a video cassette recorder (model 9500, Sony). The number of stably adherent cell on immobilized vWF was counted on images obtained at 20 randomly selected positions in the vWF-coated tubes.

Platelet Preparation and Aggregation Assay

Fresh blood from healthy volunteers was anticoagulated with 1/7 volume of ACD (2.5% trisodium citrate, 2% dextrose and 1.5% citric acid). Platelets were washed with CGS buffer (sodium chloride 0.12 M, trisodium citrate 0.0129 M and d-glucose 0.03 M, pH 6.5), resuspended in modified Tyrode's solution and allowed to incubate at 22° C. for 1 hours as previously described. In experiments using platelet-rich plasma (PRP), 1/10 volume of 3.8% trisodium citrate was used as anticoagulant. Platelet aggregation was measured using a turbidometric platelet aggregometer (Chrono-Log) at 37° C. vWF-dependent platelet aggregation was induced by addition of ristocetin or botrocetin to PRP. To differentiate CPIb-IX-mediated platelet agglutination from the second wave of integrin-dependent platelet aggregation, final concentrations of ristocetin were adjusted in each experiment to achieve a two-wave curve of aggregation. Washed platelets were used in platelet aggregation induced by a-thrombin (Enzyme Research Laboratories). To examine the effects of PKG, 100 μM of membrane permeable cGMP analogs, 8-bromo-cGMP or 8-dibutyl-cGMP (Calbiochem), or 1 μM sildenafil citrate (Pfizer) and added simultaneously with subthreshold concentrations of agonists. PKG inhibitors KT5823 (2 pM) and Rp-8PCPT-cGMP (0.2 mM) were preincubated with platelets at 37° C. for 5 minutes before addition of agonists.

Platelet Spreading

Platelets were allowed to adhere and spread on 10 μg/ml vWF-coated glass chamber slides (Nunc) at 37° C. for 1 hour. After three washes, platelets were fixed by adding 4% paraformaldehyde in PBS. Platelets were permeabilized by adding 0.1 M Tris, 0.01 M EGTA, 0.15 M NaCl, 5 mM MgCl₂, pH 7.4, containing 0.1% Triton X-100, 0.5 mM leupeptin, 1 mM PMSF, and 0.1 mM E64, and then incubated with 20 μg/ml of anti-GPIba antibody (anti-IbαC) at 22° C. for 1 hour. Platelets were further incubated with fluorescein-labeled secondary antibody at 22° C. for 30 mm. After additional washes, platelets were scanned under a Zeiss LSM 510 confocai microscope (Magnification=630×).

Measurement of CGMP Formation

Washed platelets (3×10⁸/ml) resuspended in 400 μl Tyrode's buffer were stirred at 37° C. after addition of control buffer, 1 mg/mi ristocetin alone, 15 pg/mi vWF and 1 mg/mi ristocetin, or 100 μM glyco-SNP1. The reaction was stopped by addition of 400 μl of ice-cold 12% (w/v) trichloroacetic acid. Samples were mixed and centrifuged at 2000×g for 15 ruin at 40° C. Supenatant was removed and washed with 5 volumes of water-saturated diethyl ether 4 times, and then lyophilized. cGMP levels were measured using a cGMP enzyme immunoassay kit from Amersham-Pharmacia Biotech.

### Results

PKG Promotes GPIb-IX-mediated Integrin Activation in a Reconstituted Integrin Activation Model

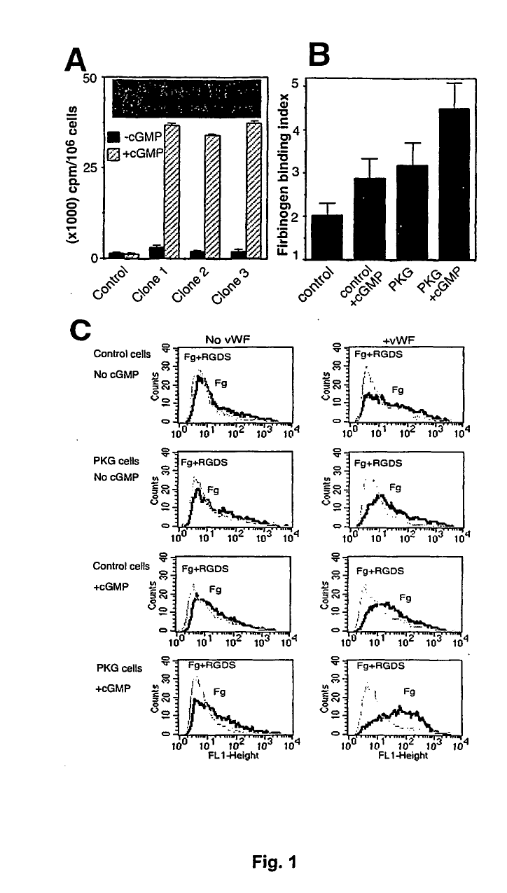

It has been shown that activation of the platelet integrin α_{iib}β₃ can be reconstituted in Chinese Hamster Ovary (CHO) cells expressing both recombinant human CPIb-IX and integrin α_{IIb}β₃ (123 cells) (see Ref. 13). In this model, binding of vWF to GPIb-IX triggers activation of integrin α_{IIb}β₃, thereby allowing specific binding of fibrinogen (a physiological ligand of integrin α_{IIb}β₃ . Preliminary studies showed that an inhibitor of PKG, i.e., KT5823, inhibited GPIb-IX-mediated integrin activation in this system (data not shown). To specifically identify the roles of PKG in GPIb-IX-mediated integrin activation, cDNAs encoding human PKG Iα or PKG Iβ was transfected into 123 cells. Results obtained with PKG Iα (FIG. 1) and PKG Iβ (not shown) were similar. Immunoblotting and PKG activity assays confirmed that human PKG I was expressed (FIG. 1A). Three different clones each of PKG Iα or Iβ-expressing cells were examined for vWF-induced integrin activation, as indicated by specific binding of soluble fibrinogen. It was found that a low concentration of vWF (12 μg/ml) induced a slight increase in fibrinogen binding to vectortransfected 123 cells. In contrast, all tested 123 cell lines expressing human PKG showed a significantly enhanced fibrinogen binding to integrin (FIGS. 1, B and C). More importantly, vWFinduced fibrinogen binding to integrin was dramatically increased in PKG-transfected cells in the presence of a membrane permeable PKG activator, 8-bromo-cCMP (FIGS. 1, B and C). Thus, recombinant human PKG stimulates GPIb-IX-mediated integrin activation.

In particular, FIG. 1 shows that expression of recombinant PKG promotes GPIb-LX-mediated integrin activation. In FIG. 1A, CHO cells expressing GPLb-JX and integrin α_{IIb}β₃ (123 cells) were transfected with cDNA encoding PKGIα or vector. Expression of PKG Ia in 123 cells was detected by Western blotting with an anti-human PKG I antibody (Insert), and recombinant PKG activity was determined in the absence or presence of 20 μM 8-bromo-cGMP as previously described. Results are expressed as mean +SD (n=3). For FIGS. 1B and 1C, reconstitution of GPIb-IX-mediated integrin activation in CHO cells was published (see Ref. 13). Integrin activation in the reconstituted system is dependent upon specific binding of vWF to GPLb-IX. PKG- or vector-transfected cells were incubated with Oregon Green-labeled fibrinogen (Fg) (20 μg/ml) and 1 mg/mi ristocetin (No cGMP) for 30 mm with (+vWF) or without (No vWF) adding 12 μg/ml vWF. These cells were also incubated with Oregon Green-labeled Fg, ristocetin and 8-bromo-cGMP (+cGMP) with or without adding vWF. Nonspecific binding was estimated by adding RGDS which inhibits fibrinogen binding to integrins (Fg+RGDS). Cells were analyzed by flow cytometry. Quantitative results from three experiments are expressed as fibrinogen binding indices (total bound fluorescence (Fg)/nonspecifically bound fluorescence (Fg+RGDS)), and shown in FIG. 1B (mean ±SD). Plots from a representative experiment are shown in FIG. 1C. Note that vWF-stimulated fibrinogen binding was significantly increased in PKG-transfected cells and was further increased by the PKG stimulator, 8-bromo-cGMP.

PKG Enhances GPIb-IX and Integrin α_{IIb}β₃-dependent Cell Adhesion to Immobilized vWF Under Flow Conditions

Under moderate to high shear rate flow conditions seen in stenotic arteries, formation of primary thrombi requires GPIb-IX-mediated transient platelet adhesion on subendotheliumimmobilized vWF, GPIb-IX-mediated integrin activation, and integrin-dependent stable platelet adhesion and aggregation (see Refs. 5-8). Thus, if PKG is important for GPIb-IX-mediated integrin activation, expression of PKG should enhance GPIb-IX- and integrin-dependent stable cell adhesion to vWF under flow conditions.

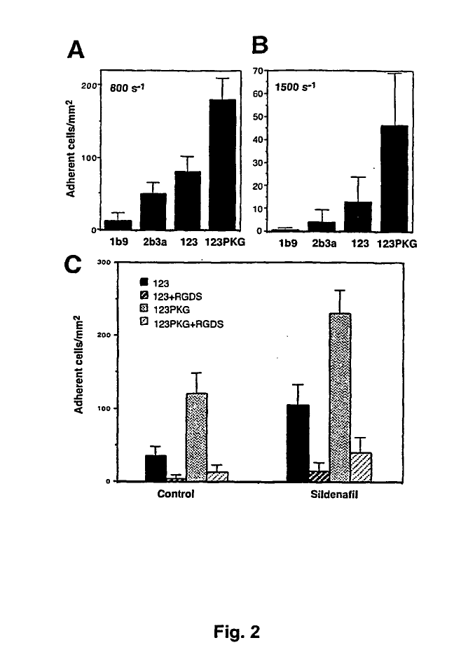

To examine this hypothesis, CHO cells expressing comparable levels of recombinant human GPIb-IX and/or integrin α_{IIb}β₃ were perfused into vWF-coated capillary tubes at shear rates (800 s⁻¹ and 1500 s⁻¹) similar to that seen in arteries. GPJb-JX-expressing cells (1b9 cells) showed only limited transient adhesion or rolling on the vWF surface, but failed to stably adhere to vWF-coated surfaces (FIGS. 2A and 2B). The cells expressing integrin α^{IIb}β₃ alone (2b3a cells) showed a low-level adhesion at 800 s⁻¹ but almost no adhesion at 1500 s⁻¹ (FIGS. 2A and 2B). The 123 cells expressing both GPJb-IX and integrin α_{IIb}β₃ showed only a small increase in stable adhesion to vWF-coated surfaces compared to 2b3a cells, indicating only a low level of GPIb-IX-mediated integrin activation (FIGS. 2A and 2B).

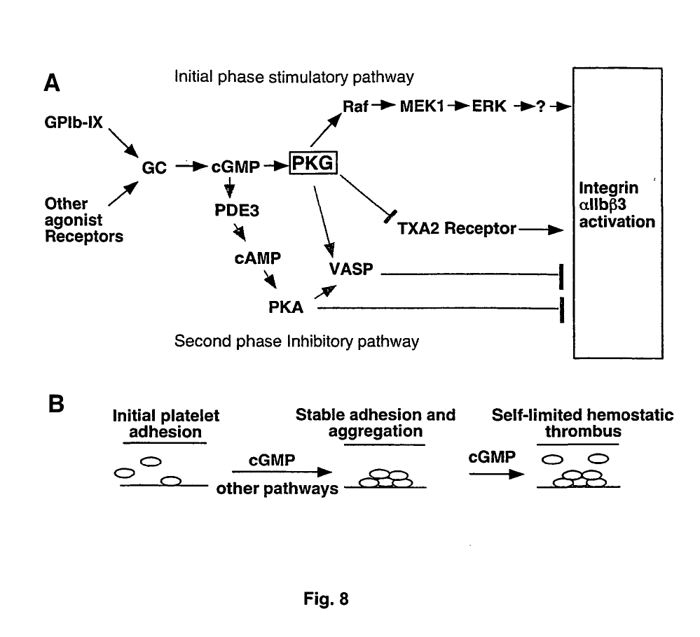

In contrast, PKG Ia-transfected 123 cells showed a markedly enhanced stable cell adhesion to vWF at both shear rates (FIGS. 2A and 2B). Stable adhesion of PKG-expressing cells was inhibited by RGDS peptides (an integrin inhibitor), indicating that PKG-stimulated stable adhesion is integrin-dependent (FIG. 2C). Furthermore, integrin-dependent adhesion of PKG-expressing 123 cells to vWF under flow conditions was enhanced by addition of a specific cGMP-enhancing drug sildenafil (FIG. 2C) or 8-bromo-cGMP (not shown). Also observed was the enhancing effects of sildenafil (FIG. 2C) on stable adhesion of 123 cells, but no effect of sildenafil on the stable adhesions of 1b9 cells or 2b3a cells under identical flow conditions was observed (not shown). Taken together, the data indicate that PKG is a stimulatory mediator of GPJb-IX-dependent integrin activation.

In particular, FIG. 2 shows that PKG enhances GPLb-IX and integrin-dependent cell adhesion under flow. In FIGS. 2A and B, vWF-coated capillary tubes were perfused with equivalent numbers (5×10⁵/ml) of CHO cells expressing recombinant GPLb-JX alone (1b9), integrin α_{IIb}β₃ alone (2b3a), both GPIb-IX and integrin α_{IIb}β₃ (123) or expressing GPJb-JX, integrin α^{IIb}β₃ and PKG Ia (123PKG) at a shear rates of 800 s⁻¹ (A) or 1500 s⁻¹ (B) for 1.2 minutes, and then perfused with the modified Tyrodes buffer for 10 minutes at the same shear rates to wash out transient adherent cells. The expenments were recorded in real time. Cells stably adherent to vWF-coated capillary tubes at 20 randomly selected locations were counted. A significantly increased stable adhesion of 123PKG cells compared to 123 cells was observed at both shear rates (P<0.0001).

In FIG. 2C, vector-transfected 123 cells (123) or 123PKG cells also was perfused into vWF-coated glass capillary tubes as described in FIG. 2A in the absence or presence (+RGDS) of an integrin inhibitor RGDS peptide (4 mM) and in the absence (Control) or presence of cGMP-enhancing drug, sildenafil (5 μM). Stable adhesion of 123PKG cells were significantly higher than vector-transfected 123 cells with or without sildenafii treatments (in all cases, P<0.0001). Sildenafil significantly increased stable adhesion of vectortransfected 123 cells or 123PKG cells (in all cases, P<0.0001). RGDS inhibited stable adhesion of 123PKG cells with or without treatment of sildenafil, and also inhibited stable adhesion of sildenafil-treated 123 cells (in all cases, P<0.0001). The experiments were repeated at least three times.

PKG is Important in GPIb-IX-Mediated Integrin Activation in Platelets

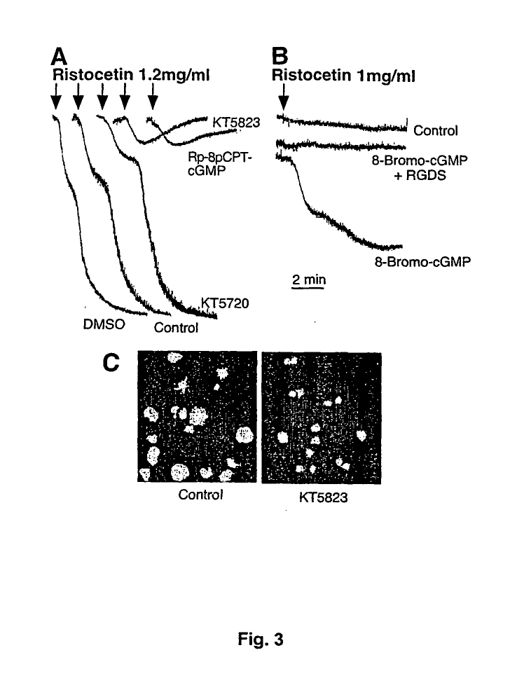

To investigate whether PKG mediates GPJb-IX-dependent integrin activation in platelets, the effect of inhibitors and stimulators of PKG on vWFinduced integrin-dependent platelet aggregation was examined. GPIb-IX on platelets does not normally bind soluble vWF, but binds to subendothelium-bound vWF at sites of vascular injury. In in vitro studies, vWF modulators, ristocetin and botrocetin, mimic the effects of subendothelial matrix to induce vWF binding to GPIb-IX. At appropriate concentrations, ristocetin-induced vWF binding to GPIb-IX causes reversible agglutination of platelets and activation of integrin α_{IIb}β₃, leading to an integrin-dependent second wave of platelet aggregation (FIG. 3A). It was found that a selective inhibitor of PKG, i.e., KT5823 (2pM), abolished the integrin-dependent second wave of platelet aggregation induced by ristocetin (FIG. 3A). The inhibitory effect of KT5823 is expected to be specific because Rp-8-pCPT-cGMP, a competitive inhibitor of cGMP binding to PKG, also inhibited ristocetin-induced second wave of platelet aggregation (FIG. 3A). In contrast, a PKA inhibitor, KT5720, had no inhibitory effects.

Similarly, the PKG inhibitors also inhibited botrocetin-induced platelet aggregation (not shown). Furthermore, membrane-permeable PKG activators, 8-bromo-cGMP (FIG. 3B) or 8-pCPT-cGMP (not shown), when added to platelets together with a subthreshold concentration (1 mg/mm) of ristocetin (or botrocetin), induced integrin-dependent platelet aggregation. In addition, KT5823 also inhibited platelet spreading on a vWF-coated surface (FIG. 3C) which requires GPIb-IX-induced integrin activation (see Refs. 7, 13, and 14). These data indicate that PKG indeed mediates GPIb-IX-induced integrin activation in platelets.

In particular, FIG. 3 shows effects of PKG inhibitors and activators on integrin-dependent platelet aggregation induced by vWF-GPIb-IX interaction. In FIG. 3A, platelet rich-plasma (PRP) was preincubated at 37° C. for 5 mm with PKG inhibitors KT5823 (2 pM) or Rp-8pCPT-cGMP (0.2 μM). PRP was also incubated with DMSO (vehicle for KT5823), a PKA inhibitor, KT5720 (2 pM), or buffer (Control). The vWF modulator, ristocetin, was then added to induce vWF-GPIb-IX interaction. Ristocetin-induced platelet aggregation was recorded using a platelet aggregometer. In FIG. 3B, a subthreshold concentration of ristocetin was added to PRP immediately followed by addition of 8-bromo-cGMP or buffer (Control). PRP also was preincubated with integrin inhibitor RGDS before adding ristocetin and 8-bromo-cGMP. In FIG. 3C, platelets were preincubated with or without PKG inhibitor KT5823, then allowed to spread on vWF-coated glass chamber slides. Platelets were stained with an anti-GPIb antibody, and fluorescein-labeled secondary antibody. The slides were imaged under a confocal microscope (magnification =630×).

vWF Induces an Increase in Platelet cGMP Levels

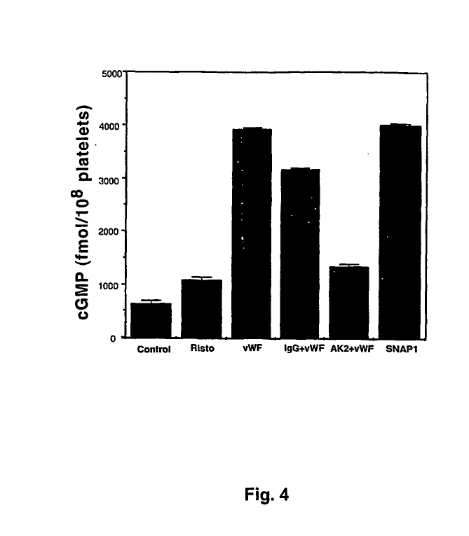

PKG activity is regulated by intracellular cCMP levels. Thus, if PKG is activated downstream of CPIb-IX, ligand binding to GPIb-IX is expected to stimulate an increase in intracellular cCMP levels. Indeed, there was a dramatic increase in platelet cCMP following ristocetin-induced vWF binding to GPIb-IX, and vWF-induced increase in cGMP levels was inhibited by anti-GPIba monoclonal antibodies but not control IgG (FIG. 4). The cGMP levels in vWF-stimulated platelets were comparable to that stimulated with glyco-SNAP1, a compound that releases nitric oxide and thus activates PKG) (FIG. 4). Thus, ligand binding to CPIb-IX increases the intracellular cCMP level to an extent that is capable of activating PKG.

FIG. 4 shows a vWF-induced increase in intra-platelet cGMP. Washed platelets (3×108/ml) were preincubated at 37° C. for 10 mm with buffer, control JgG, a monoclonal antibody against GPIba, AK2. Platelets then were further incubated in a platelet aggregometer for 5 minutes after addition of buffer (Control), 1.25 mg/ml ristocetin only (Risto), ristocetin plus vWF (vWF) (15 μg/ml) or glyco-SNAP1 (100 μM). The reaction was stopped by addition of 400 μl of ice-cold 12% (w/v) trichloroacetic acid. CGMP concentrations were determined using a cGMP enzyme immunoassay kit. Results are expressed as mean ±SD (n=3).

The Roles of PKG in Low Dose Thrombin-Induced Platelet Aggregation

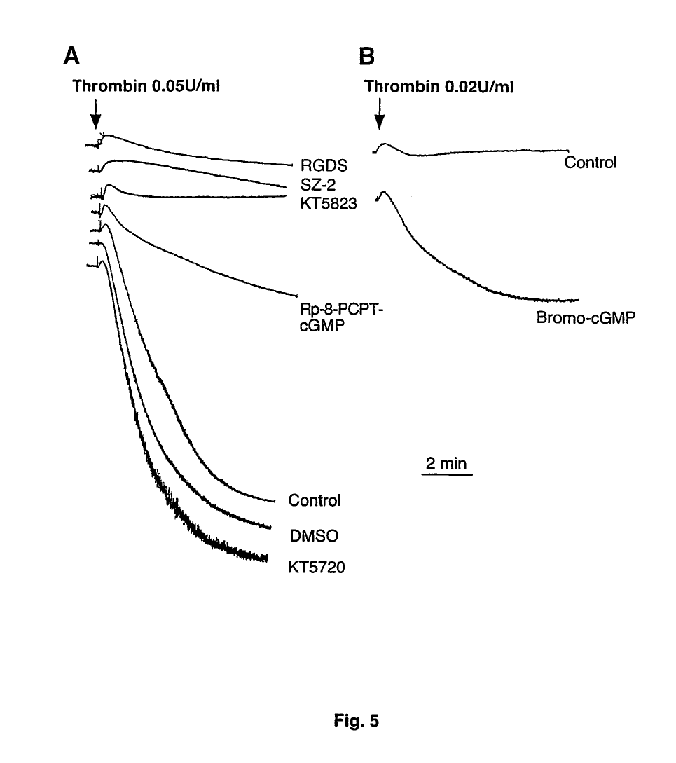

It is known that GPIb-IX is required for low-dose thrombin-induced platelet aggregation (see Refs. 18-21). To study whether PKG also promotes low dose thrombin-induced integrin activation, 0.05 units/ml of α-thrombin was used to induce platelet aggregation. Under these conditions, platelet aggregation was inhibited by RGDS indicating its dependence on integrin activation. Platelet aggregation also was inhibited by SZ2, a monoclonal antibody directed against the thrombin binding region of GPIb a chain (GPIbα) (FIG. 5A), but not by control IgG (not shown). This is consistent with previous findings using GPTb-IX-deficient platelets from Bernard-Soulier syndrome patients (see Ref. 18) and monoclonal antibodies (see Refs. 19, 20, and 49). KT5823 (2 μM) and Rp-8-pCPT-cGMP inhibited platelet aggregation induced by 0.05 units/ml thrombin (FIG. 5A). Furthermore, the membrane-permeable CGMP analog, 8-bromo-cGMP, when added to platelets together with a subthreshold concentration (0.02 units/ml) of thrombin, induced platelet aggregation (FIG. 5B). These data indicate that PKG promotes platelet activation induced by low doses of thrombin.

FIG. 5 shows effects of PKG inhibitors and activators on thrombin-induced platelet aggregation. In FIG. 5A, washed platelets were preincubated with 40 pg/mi anti-GPIba monoclonal antibody SZ2, integrin inhibitor RGDS, or inhibitors of PKG, KT5823 (2 μM) or Rp-8pCPT-cGMP (0.2 mM) (Calbiochem), for 5 min. Platelets also were preincubated with DMSO (vehicle for KT5823), KT5720 or buffer (Control) as controls. α-Thrombin (0.05 units/mi) then was added to induce platelet aggregation. Nonspecific JgG and RGES peptides also were examined as additional controls, but had no effect (not shown). In FIG. 5B, a subthreshold dose of thrombin was added to platelets immediately followed by addition of PKG activator 8-bromo-cGMP or buffer (Control). Shown in the figure are the representative results of at least three experiments.

Effects of cGMP-Enhancing Drug Sildenafil on Platelet Activation

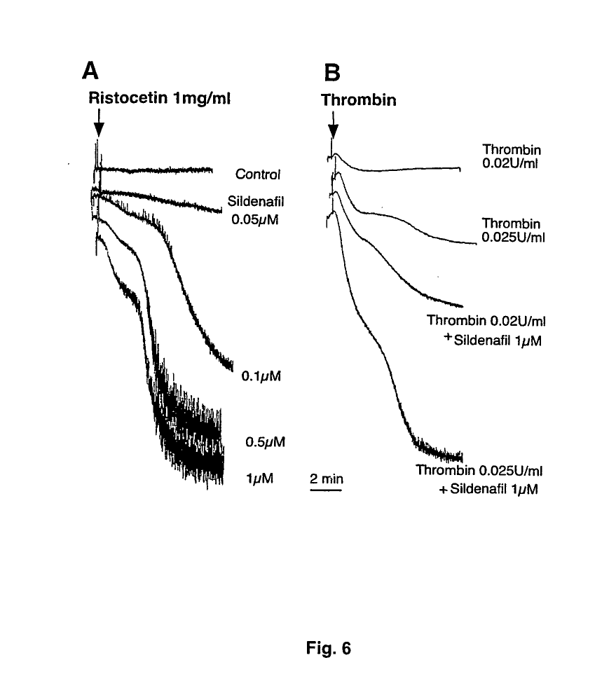

A commercial drug used for treatment of erectile dysfunction, sildenafil, increases intracellular cGMP and activates PKG. Thus, if PKG stimulates platelet activation, sildenafil is expected to be stimulatory. Indeed, addition of sildenafil to platelets induces platelet aggregation together with subthreshold concentrations of either ristocetin (FIG. 6A) or thrombin (FIG. 6B). Because sildenafil functions by specifically increasing endogenous cGMP, this result further excludes the possibility of nonspecific effects of exogenous cGMP analogs and supports the conclusion that the cGMP-PKG pathway stimulates platelet (integrin) activation. Furthermore, this result provides direct evidence that sildenafil may potentiate platelet activation.

FIG. 6 shows effects of sildenafil on platelet aggregation. In FIG. 6A, increasing concentrations of sildenafil or control 0.15 M NaCl solution were added to platelet-rich plasma followed by a subthreshold concentration of ristocetin. Platelet aggregation was recorded using a platelet aggregometer. In FIG. 6B, sildenafil or control buffer was added to washed platelets followed by addition of subthreshold doses of thrombin to induce platelet aggregation. It was noted that platelet aggregation occurs in the presence of sildenafil. The figure includes the representative results of at least three experiments.

cGMP Induces Biphasic Platelet Responses

The stimulatory role of cGMP-PKG pathway in CPIb-IX-dependent platelet integrin activation appears to contradict the concept that cGMP inhibits platelet activation. However, it was noted that a major difference exists in the timing of cGMP elevation between the present experiments and previous studies. Thus, the relationship between the timing of cGMP elevation and its effects on platelets was examined.

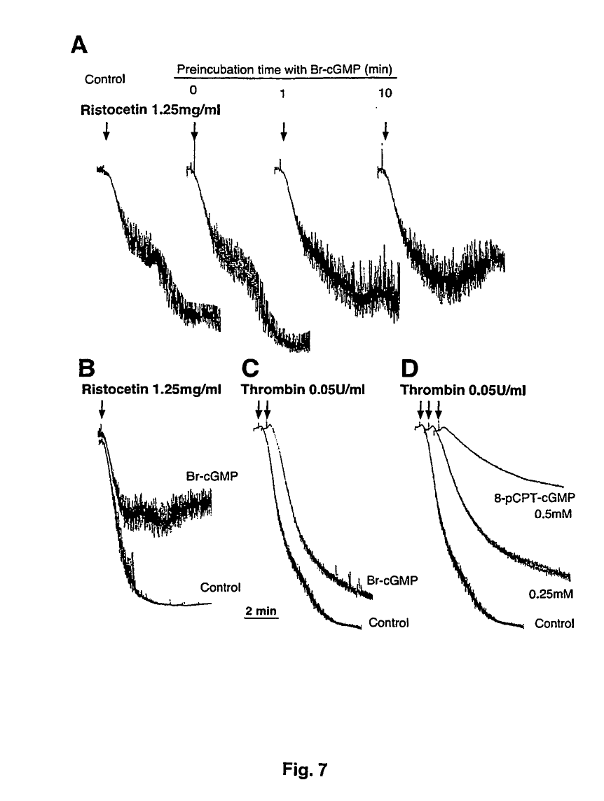

The addition of 8-bromo-cGMP (100 μM) simultaneously with the platelet agonist (ristocetin (FIG. 7A) or thrombin (FIG. 5)) promoted GPIb-IX-dependent platelet activation (FIG. 7A, also see FIGS. 3 and 5). However, when platelets were preincubated with cGMP for increasing lengths of time, the stimulatory effect was diminished, and cGMP became increasingly inhibitory. The inhibitory effect becomes significant after 10 minutes preincubation with cGMP. The preincubation time for the inhibitory effects of cGMP may vary in different experiments. In some experiments, 30 minutes preincubation time was required for a slight inhibitory effect (not shown). The preincubation time required for cGMP to inhibit platelets was shortened when cGMP concentration was increased. At 1 mM 8-bromo-cGMP or 0.5 mM 8-pCPT-cCMP, the preincubation time was shortened to less than 5 minutes. This is consistent with published data that a prolonged preincubation with cCMP or a high concentration (500-3000 μM) of cGMP is required to inhibit platelet aggregation. Thus, the apparently paradoxical effects of cGMP are in fact a biphasic effect associated with the timing and levels of cGMP elevation.

In particular, FIG. 7 shows a time-dependent biphasic effects of cGMP on GPLb-IX-dependent platelet aggregation. In FIG. 7A, PRP was preincubated with 0.1 mM 8-bromo-cGMP for increasing lengths of time at 37° C. as indicated. The vWF modulator, ristocetin (1.25 mg/ml), then was added to induce platelet aggregation in a turbidometric platelet aggregometer. In FIG. 7B, PRP was preincubated with a high concentration of 8-bromo-cGMP (1 mM) for 5 minutes. Ristocetin then was added to induce platelet aggregation. In FIGS. 7C and 7D, washed platelets in modified Tyrode's solution were preincubated with 1 mM 8-bromo-cGMP (C), or various concentrations of 8-pCPT-cGMP (D) at 37° C. for 5 minutes. Thrombin (0.05 u/ml) then was added to induce platelet aggregation.

FIG. 8 illustrates a new concept of cGMP signaling in platelet activation and hemostasis. In FIG. 8A, GPIb-IX (and other agonists) induces elevation of cGMP and activation of PKG which mediates initial phase integrin activation signal via the ERK pathway (see Ref. 23). Subsequently, cGMP induces a second phase negative regulatory signal potentially via several pathways: (1) PKG-mediated TXA2 receptor phosphorylation, (2) VASP phosphorylation, and (3) activation of the PKA pathway (see Refs. 27, 35, 36, 57, and 58).

In FIG. 8B, upon vascular injury, platelets adhere to the exposed subendothelial matrix via GPIb-IX interaction with vWF. This interaction also induces cGMP elevation, which promotes platelet activation and formation of hemostatic thrombi. Platelet activation can also be induced by other agonists such as collagen, thrombin and ADP. Continued cGMP elevation in aggregated platelets induces second phase inhibitory cGMP signaling resulting in inhibition of further recruitment of platelets from blood flow. This prevents overgrowth of hemostatic thrombi (see Ref. 56), thus reducing the probability of thrombosis in normal person. However, in patients at high thrombotic risk, initial phase of cGMP-promoted thrombus formation may be sufficient to cause occlusion of blood vessels leading to thrombosis.

The above-described tests show that cGMP-PKG signaling pathway can play a stimulatory role in GPIb-IXdependent activation of platelet integrin α_{IIb}β₃. This conclusion is based on data showing that (1) expression of recombinant PKG promotes integrin activation induced by vWF binding to GPIb-IX, and enhances GPIb-IX- and integrin-dependent cell adhesion to vWF; (2) integrin-dependent platelet aggregation induced by either low-dose thrombin or vWF is blocked by PKG inhibitors and enhanced by cGMP analogs; (3) the cGMP-enhancing drug sildenafil also potentiates GPIbJX-dependent integrin activation; and (4) ligand binding to GPIb-IX increases intracellular cGMP levels. In addition, the conclusion that PKG stimulates platelet activation is also supported by a finding that PKG is required for the GPIb-IX-mediated activation of ERK pathway, which is important in GPIb-IX-dependent integrin activation (see Ref. 23). These data are derived from experiments using either the molecular biological technique in the reconstituted CHO cell model of integrin activation or pharmacological studies in human platelets.

The use of recombinant DNA techniques excludes the possibility of nonspecific effects of pharmacological reagents, and studies in human platelets indicates that the results obtained in the CHO cell model are relevant to platelet function. Also, the use of a combination of several PKG inhibitors and activators with different mechanisms of action in human platelets also minimizes the chance that potential nonspecific effects of a drug could influence the outcome of our studies. Thus, the data reveals a novel role for PKG in stimulating integrin activation.

It is important that the stimulatory role of PKG manifests only when platelets are exposed simultaneously to the agonists, α-thrombin or ristocetin. Elevation of cGMP alone is not sufficient to activate integrins (not shown). Thus, it appears that the cGMPPKG pathway is not the only pathway required for GPIb-IX-mediated integrin activation. In this respect, it recently has been shown that vWF-induced platelet activation involves coordination of different signaling pathways, and requires the Fc receptor γII/Fc receptor γ-chain and Syk signaling pathway (see Refs. 52-55). It is common that platelet activation induced by an agonist requires more than one parallel signaling pathways. For example, low dose thrombin requires both GPIb-IX and hepatohelical receptor-coupled signaling pathways, collagen stimulates both glycoprotein VI and integrin α2β1, and ADP requires both Gi- and Gq-coupled receptor pathways in order to be sufficient to activate platelets.

The stimulatory roles of PKG appear to be specially important in GPIb-IX-dependent integrin activation pathway but may not be required for some other platelet activation pathways, because it was found that PKG inhibitors had no effect on platelet aggregation induced by collagen (not shown). This is consistent with a previous report that platelets from PKG I-deficient mice were not different from the wild type mice in aggregation responses to collagen (see Ref. 51). This, however, does not exclude the possible involvement of cGMP-PKG-ERK pathway as one of the parallel stimulatory signaling pathways induced by the CPIb-IX-independent agonists, because cGMP levels and ERK kinase activities are known to be elevated by these agonists. It would be interesting to further investigate if there is cross talks between the GPTb-JX-dependent integrin activation pathway and signaling pathways of GPIb-IX-independent platelet agonists (see Refs. 32 and 33).

It previously was established that preincubation of platelets with cGMP analogs or NO donors inhibit subsequent platelet activation induced by collagen, ADP, and thrombin. Thus, it is apparent that the finding of a stimulatory role of cGMP contradicts the established concept. However, there is a major difference between the present experiments and previously published experiments. In all previous publications showing the inhibitory effects of CGMP, cGMP analogs were allowed to preincubate with platelets before addition of platelet agonists. The lower the cGMP concentration, the longer the preincubation time (5 minutes preincubation when using high concentrations (0.5-2 mM) of cGMP analogs, or 15-30 minutes preincubation when using lower concentrations of cGMP (0.1-0.5 mM) (see Refs. 50 and 51). Also, cGMP-induced platelet inhibition requires a longer period of preincubation than high concentrations of NO donors, suggesting that the inhibitory effects of NO donors may involve either a higher peak of cCMP elevation or other mechanisms in addition to cGMP elevation. The possible involvement of a different mechanism in NO-induced platelet inhibition is supported by the finding that NO-induced platelet inhibition is reversed by PKA inhibitors, and that some NO donors may have direct negative effect on integrin function (see Refs. 35-37).

The data shows that CGMP (0.1 mM) is stimulatory only when added simultaneously with or immediately after the addition of platelet agonists. In contrast, after prolonged preincubation or with high concentrations of cGMP, cCMP becomes inhibitory to GPIb-IX-dependent platelet aggregation (FIG. 7). Thus, it appears that whether cGMP has stimulatory or inhibitory effect on GPIb-IX-dependent platelet activation is determined by the timing of cGMP elevation and by cGMP concentrations. This finding explains the apparent paradoxical effects of cGMP on platelet activation, and suggests that the roles of cGMP in platelet activation are biphasic. The biphasic role of cGMP-PKG pathway is strongly supported by our data that PKG-dependent activation of ERK during platelet activation is a transient event which peaks within 0.5-1 minute and becomes quickly diminished (see Ref. 23). Furthermore, it previously was shown that cCMP elevation induced by endogenous platelet NO (a low concentration compared to exogenous NO donors) had no inhibitory effect on primary platelet activation, but, after a delay, inhibited recruitment of subsequently added platelets to already formed aggregates. Also, although platelet aggregation in response to collagen was not affected in PKG-deficient mice, PKG deficiency affected platelet inhibition induced by the prolonged preincubation with cGMP (see Refs. 51 and 56).

Thus, the inhibitory role of cGMP-PKG pathway under physiological conditions is a delayed secondary event. Interestingly, it was shown that, while platelet adhesion and thrombus formation occurs normally at the site of ischemia/reperfusion injury in wild type mice, more platelets appear to be recruited to the site of ischemia/reperfusion injury in PKG-deficient mice. Thus, the biphasic response of platelets to cGMP elevation is of physiological significance because this effect not only mediates rapid activation of platelets upon vascular injury and formation of primary thrombus, but also serve to inhibit the overgrowth of thrombus so that severe thrombosis would be less likely to occur during normal hemostasis (FIG. 8).

The finding that cGMP induces biphasic platelet responses is important not only to the understanding of the platelet physiology, but also important to pharmacology and therapeutic applications of the cGMP enhancing drugs. The specific cGMP-enhancing drug, sildenafil, increases intracellular cGMP levels by inhibiting PDE5, and thus causes vasodilation. PDE5 is a major phosphodiesterase in platelets (see Ref. 24). According to the current theory that cGMP inhibit platelets, sildenafil should inhibit platelet function in a way similar to NO donors. However, it was found that sildenafil did not inhibit platelet aggregation in vitro (not shown), which is consistent with the previous report that sildenafil by itself did not inhibit platelet activation induced by any tested agonists (ADP, collagen, A23 187, U46619, and platelet activating factor) (see Ref. 38). On the contrary, use of sildenafil has been reportedly associated with severe thrombotic events (myocardial infarction and cerebral thrombosis) in some patients, which cannot be satisfactorily explained by the current concept of cGMP signaling (see Refs. 39-42). It was shown that although sildenafil by itself is not sufficient to cause platelet activation, this drug enhances platelet activation in the presence of subthreshold concentrations of platelet agonists such as thrombin and vWF that are likely to be present at sites of vascular injury or atherosclerotic lesions. Thus, sildenafil potentially may increase thrombotic risks in patients with preexisting thrombotic conditions such as artherosclerosis. This provides a mechanistic reason for caution when prescribing sidenafil or other specific cGMP-enhancing drugs to patients at thrombotic risk.

Modifications and variations of the invention as hereinbefore set forth can be made without departing from the spirit and scope thereof, and, therefore, only such limitations should be imposed as are indicated by the appended claims.

### References

1. Ginsberg M H, Du X, Plow E F: Inside-out integrin signalling. Curr. Opin. Cell Biol. 4:766-771, 1992

2. Phillips D R, Charo I F, Scarborough R M: GPIIb-IIIa: the responsive integrin. Cell 65:359-362, 1991

3. Shattil S J, Kashiwagi H, Pampori N: Jntegrin signaling: the platelet paradigm. Blood 91:2645-2657, 1998

4. Weiss H J, Turitto V T, Baumgartner H R: Effect of shear rate on platelet interaction with subendothelium in citrated and native blood. I. Shear rate dependent decrease of adhesion in von Willebrand's disease and the Bernard-Soulier syndrome. J. Lab. Clin. Med. 92:750-764, 1978

5. Sakariassen K S, Bolhuis P A, Sixma J J: Human blood platelet adhesion to artery subendothelium is mediated by factor Vhf-von Willebrand factor bound to the subendothelium. Nature 279:636-638,

6. Sakariassen K S, Nievelstein P F, Coller B S, Sixma J J: The role of platelet membrane glycoproteins Ib and IIb-IIIa in platelet adherence to human artery subendothelium. Br J Haematol 63:681-691, 1986

7. Savage B, Saldivar E, Ruggeri Z M: Initiation of platelet adhesion by arrest onto fibrinogen or translocation on von Willebrand factor. Cell 84:289-297, 1996

8. Savage B, Almus-Jacobs F, Rugged Z M: Specific synergy of multiple substrate-receptor interactions in platelet thrombus formation under flow. Cell 94:657-666, 1998

9. Du X, Ginsberg M H: Signaling and platelet adhesion. Advances in Molecular and Cell Biology 28:269-301, 1999

10. De Marco L, Girolami A, Russell 5, Rugged Z M: Interaction of asialo von Willebrand factor with glycoprotein Ib induces fibrinogen binding to the glycoprotein ILb/Illa complex and mediates platelet aggregation. J Clin Invest 75:1198-1203, 1985

11. Gralnick H R, Williams S B, Coller B S: Asialo von Willebrand factor interactions with platelets. Interdependence of glycoproteins Lb and hLb/HIa for binding and aggregation. J Clin Invest 75:19-25, 1985

12. Kroll M H, Harris T S, Moake J L, Handin R I, Schafer Al: von Willebrand factor binding to platelet GpLb initiates signals for platelet activation. J Clin Invest 88:1568-1573, 1991

13. Gu M, Xi X, Englund G D, Bemdt M C, Du X: Analysis of the roles of 14-3-3 in the platelet glycoprotein Ib-IX-mediated activation of integrin α_{IIb}β₃ using a reconstituted mammalian cell expression model. J. Cell. Biol. 147:1085-1096, 1999

14. Savage B, Shattil S J, Rugged Z M: Modulation of platelet function through adhesion receptors. A dual role for glycoprotein IIb-IIIa (integrin alpha IIb beta 3) mediated by fibrinogen and glycoprotein Lb-von Willebrand factor. J Biol Chem 267:11300-11306, 1992

15. Okumura T, Jamieson G A: Platelet glycocalicin: A single receptor for platelet aggregation induced by thrombin or ristocetin. Thromb. Res. 8:701-706., 1976

16. Harmon J T, Jamieson G A: The glycocalicin portion of platelet glycoprotein Lb expresses both high and moderate affinity receptor sites for thrombin. A soluble radioreceptor assay for the interaction of thrombin with platelets. J Biol Chem 261:13224-13229, 1986

17. Ward C M, Andrews R K, Smith A L, Berndt M C: Mocarhagin, a novel cobra venom metalloproteinase, cleaves the platelet von Willebrand factor receptor glycoprotein Lbalpha. Identification of the sulfated tyrosine/anionic sequence Tyr-276-Glu-282 of glycoprotein Ibalpha as a binding site for von Willebrand factor and alpha-thrombin. Biochemistry 35:4929-4938, 1996

18. Jamieson G A, Okumura T: Reduced thrombin binding and aggregation in BernardSoulier platelet. J. Clin. Invest. 61:861-864, 1978

19. Yamamoto N, Kitagawa H, Tanoue K, Yamazaki H: Monoclonal antibody to glycoprotein Ib inhibits both thrombin- and ristocetin-induced platelet aggregations. Thromb Res 39:751-759, 1985

20. Mazzucato M, Marco L D, Masotti A, Pradella P. Bahou W F, Rugged Z M: Characterization of the initial alpha-thrombin interaction with glycoprotein Lb alpha in relation to platelet activation. J Biol Chem 273:1880-1887, 1998

21. Greco N J, Tandon N N, Jones G D, Kornhauser R, Jackson B, Yamamoto N, Tanoue K, Jamieson G A: Contributions of glycoprotein Ib and the seven transmembrane domain receptor to increases in platelet cytoplasmic [Ca2+] induced by alpha-thrombin. Biochemistry 35:906-914, 1996

22. Lopez J A, Andrews R K, Afshar-Kharghan V. Berndt M C: Bernard-Soulier syndrome. Blood 91:4397-4418, 1998

23. Li Z, Xi X, Du X: A mitogen-activated protein kinase-dependent signaling pathway in the activation of platelet integrin α_{IIb}β₃ J Biol Chem 276(45):42226-42232, 2001

24. Haslam R J, Dickinson N T, Jang E K: Cyclic nucleotides and phosphodiesterases in platelets. Thromb Haemost 82:412-423, 1999

25. Butt E, Geiger J, Jarchau T, Lohmann S M, Walter U: The cGMP-dependent protein kinase—gene, protein, and function. Neurochem Res 18:27-42, 1993

26. Smolenski A, Bachmann C, Reinhard K, Honig-Liedl P. Jarchau T, Hoschuetzky H, Walter U: Analysis and regulation of vasodilator-stimulated phosphoprotein serine 239 phosphorylation in vitro and in intact cells using a phosphospecific monoclonal antibody. J Biol Chem 273:20029-20035,

27. Wang G R, Zhu Y, Halushka P V, Lincoln T M, Mendelsohn M E: Mechanism of platelet inhibition by nitric oxide: in vivo phosphorylation of thromboxane receptor by cyclic GMPdependent protein kinase. Proc Natl Acad Sci U S A 95:4888-4893, 1998

28. Smolenski A, Poller W, Walter U, Lohmann S M: Regulation of human endothelial cell focal adhesion sites and migration by cGMP-dependent protein kinase I. J Biol Chem 275:25723-25732, 2000

29. Furchgott R F, Vanhoutte P M: Endothelium-derived relaxing and contracting factors. Faseb J 3:2007-20 18, 1989

30. McDonald U, Murad F: Nitric oxide and cyclic GMP signaling. Proc Soc Exp Biol Med 211:1-6, 1996

31. Corbin J D, Francis S H: Cyclic GMP phosphodiesterase-5: target of sildenafil. J Biol Chem 274:13729-13732, 1999

32. Chiang T M, Beachey E H, Kang A H: Interaction of a chick skin collagen fragment (alphal -CBS) with human platelets. Biochemical studies during the aggregation and release reaction. J Biol Chem 250:6916-6922, 1975

33. Haslam R J, Davidson M M, McClenaghan M D: Cytochalasin B, the blood platelet release reaction and cyclic GMP. Nature 253:455-457, 1975

34. Chiang T M, Dixit S N, Kang A H: Effect of cyclic 3′,5′-guanosine monophosphate on human platelet function. J Lab Clin Med 88:215-221, 1976

35. Maurice D H, Haslam R J: Molecular basis of the synergistic inhibition of platelet function by nitr-ovasodilators-and activators of adeny-late cyclase: inhibition of cyclic AMP breakdown by cyclic GMP. Mol Pharmacol 37:671-681, 1990

36. Azula F J, Alzola E S, Conde M, Trueba M, Macarulla J M, Marino A: Thrombinstimulated phospholipase C activity is inhibited without visible delay by a rapid increase in the cyclic GMP levels induced by sodium nitroprusside. Mol Pharinacol 50:367-379, 1996

37. Yan B, Smith J W: A redox site involved in integrin activation. J Biol Chem 275:39964-39972, 2000

38. Wallis R M, Corbin J D, Francis S H, Ellis P: Tissue distribution of phosphodiesterase families and the effects of sildenafil on tissue cyclic nucleotides, platelet function, and the contractile responses of trabeculae carneae and aortic rings in vitro. Am J Cardiol 83:3C-12C, 1999

39. Feenstra J, van Drie-Pierik R J, Lade C F, Stricker B H: Acute myocardial infarction associated with sildenafil. Lancet 352:957-958, 1998

40. Arora R R, Timoney M, Melilli U: Acute myocardial infarction after the use of sildenafil. N Engl J Med 341:700, 1999

41. Porter A, Mager A, Birnbaum Y, Strasberg B, Sclarovsky 5, Rechavia E: Acute myocardial infarction following sildenafil citrate (Viagra) intake in a nitrate-free patient. Clin Cardiol 22:762-763, 1999

42. Mitka M: Some men who take Viagra die—why? Jama 283:590, 593, 2000

43. Sandberg M, Natarajan V, Ronander I, Kalderon D, Walter U, Lohmann S M, Jahnsen T: Molecular cloning and predicted full-length amino acid sequence of the type I beta isozyme of cGMP-dependent protein kinase from human placenta. Tissue distribution and developmental changes in rat. FEBS Lett 255:321-329, 1989

44. Browning D D, McShane M P, Marty C, Ye RD: Nitric oxide activation of p38 mitogen-activated protein kinase in 293T fibroblasts requires cGMP-dependent protein kinase. J Biol Chem 275:28 1 1-28 16, 2000

45. Colbran J L, Francis S H, Leach A B, Thomas M K, Jiang H, McAllister L M, Corbin J D: A phenylalanine in peptide substrates provides for selectivity between cGMP- and cAMP-dependent protein kinases. J Biol Chem 267:9589-9594, 1992

46. Du X, Plow E F, Frelinger ALl, O'Toole T E, Loftus J C, Ginsberg M H: Ligands “activate” integrin α_{IIb}β₃ (platelet GPILb-IIIa) . Cell 65:409-416,1991

47. Du X, Fox J E, Pei 5: Identifcation of a binding sequence for the 14-3-3 protein within the cytoplasmic domain of the adhesion receptor, platelet glycoprotein Lba. J Biol Chem 271:7362-7367, 1996

48. Ruan C G, Du XP, Xi XD, Castaldi P A, Berndt M C: A murine antiglycoprotein Ib complex monoclonal antibody, SZ 2, inhibits platelet aggregation induced by both ristocetin and collagen. Blood 69:570-577, 1987

49. Mazurov A V, Vinogradov D V, Vlasilc T N, Repin V S. Booth W J, Berndt M C: Characterization of an antiglycoprotein Ib monoclonal antibody that specifically inhibits plateletthrombin interaction. Thromb Res 62:673-684, 1991

50. Mellion B T, Ignarro U, Ohlstein E H, Pontecorvo E G, Hyman A L, Kadowitz P J: Evidence for the inhibitory role of guanosine 3′, 5′-monophosphate in ADP-induced human platelet aggregation in the presence of nitric oxide and related vasodilators. Blood 57:946-955, 1981

51. Massberg 5, Sausbier M, Klatt P. Bauer M, Pfeifer A, Siess W, Fassler R, Ruth P, Krombach F, Hofmann F: Increased adhesion and aggregation of platelets lacking cyclic guanosine 3′,5′-monophosphate kinase I. J Exp Med 189:1255-1264, 1999

52. Canobbio I, Bertoni A, Lova P, Paganini 5, Hirsch E, Sinigaglia F, Balduini C, Torti M: Platelet activation by von willebrand factor requires coordinated signaling through thromboxane A2 and FCγIIA receptor. J Biol Chem 276:26022-26029, 2001

53. Yanabu M, Ozaki Y, Nomura 5, Miyake T, Miyazaki Y, Kagawa H, Yamanaka Y, Asazuma N, Satoh K, Kume 5, Komiyama Y, Fukuhara 5: Tyrosine phosphorylation and p72syk activation˜by˜an antbglycoprotein Ib monoclonal antibody. Blood 89: 1590-1598, 1997

54. Sullam P M, Hyun W C, Szollosi J, Dong J, Foss W M, Lopez JA: Physical proximity and functional interplay of the glycoprotein Ib-IX-V complex and the Fe receptor FcgammaRllA on the platelet plasma membrane. J Biol Chem 273:5331-5336., 1998

55. Wu Y, Suzuki-Inoue K, Satoh K, Asazuma N, Yatomi Y, Berndt M C, Ozaki Y: Role of Fc receptor gamma-chain in platelet glycoprotein Ib-mediated signaling. Blood 97:3836-3845., 2001

56. Freedman J E, Uoscalzo J, Barnard M R, Alpert C, Keaney I F, Michelson A D: Nitric oxide released from activated platelets inhibits platelet recruitment. J Clin Invest 100:350-356, 1997

57. Aszodi A, Pfeifer A, Ahmad M, Glauner M, Zhou X H, Ny U, Andersson K E, Kehrel B, Offermanns 5, Fassler R: The vasodilator-stimulated phosphoprotein (VASP) is involved in cGMP- and cAMP-mediated inhibition of agonist-induced platelet aggregation, but is dispensable for smooth muscle function. Embo J 18:37-48, 1999

58. Hauser W, Knobeloch K P, Eigenthaler M, Gambaryan 5, Krenn V, Geiger J, Glazova M, Rohde E, Horak I, Walter U, Zimmer M: Megakaryocyte hyperplasia and enhanced agonist-induced platelet activation in vasodilator-stimulated phosphoprotein knockout mice. Proc Natl Acad Sci U S A 96:8120-8125, 1999.

What is claimed is:

1. A method of treating a thrombotic disease or condition comprising a step of administering a therapeutically effective amount of an active agent that, directly or indirectly, inhibits production of guanosine 3′,5′-cyclic monophosphate to a mammal in need of such treatment.

2. The method of claim 1 wherein the disease or condition is selected from the group consisting of myocardial infarction, cerebral thrombosis, arterial thrombosis, and occlusion of blood vessels.

3. The method of claim 1 wherein the active agent is selected from a group consisting of:
(a) a compound that inhibits production of guanosine 3′,5′-cyclic monophosphate (cGMP) in platelets;
(b) a compound that stimulates cGMP-specific phosphodiesterase;
(c) a compound that inhibits activity of protein kinase G;
(d) a compound that inhibits activity of the cRaf-MEK-ERK pathway or p38 pathway;
(e) a mixture of two or more active agents defined in groups (a), (b), (c), and (d).

4. The method of claim 1 wherein the active agent is selected from the group consisting of a guanylyl cyclase inhibitor, a phosphodiesterase activator, a protein kinase G inhibitor, an inhibitor of cRaf-MEK-ERK pathway, an inhibitor of the p38 pathway, and mixtures thereof.

5. The method of claim 4 wherein the guanylyl cyclase inhibitor is selected from the group consisting of Ly83583, methylene blue, NS2028, ODQ, zinc (II) protoporphyrin, and mixtures thereof.

6. The method of claim 4 wherein the protein kinase G inhibitor is selected from the group consisting of KT5823, Rp-cGMP, Rp-8-bromo-cGMP, Rp-8-pCPT-cGMP, and mixtures thereof.

7. The method of claim 4 wherein the inhibitor of the cRaf-MEK-ERK or p38 pathways is selected from the group consisting of PD98059, U0125, U0126, PD184352, Apigenin, ZM336372, SB203580, PD169316, SB220025, SC68376, SFK-86002, and SB202190.

8. A method of treating a hemostatic disease comprising a step of administering a therapeutically effective amount of an active agent that, directly or indirectly, stimulates production of guanosine 3′,5′-cyclic monophosphate to a mammal in need of such treatment.

9. The method of claim 8 wherein the hemostatic condition is selected from the group consisting of von Willebrand's disease, thrombocytopenia, and a functional platelet disorder.

10. The method of claim 8 wherein the active agent is selected from the group consisting of:
(a) a compound for stimulating production of guanosine 3′,5′-cyclic monophosphate;
(b) a compound that inhibits cGMP-specific phosphodiesterase;
(c) a compound that stimulates activity of a protein kinase G;
(d) a compound that enhances activity of cRaf-MEK-ERK pathway or p38 pathway;
(e) a mixture of two or more active agents defined in groups (a), (b), (c), and (d).

11. The method of claim 8 wherein the active agent is selected from the group consisting of a guanylyl cyclase activator, a protein kinase G activator, a cGMP-specific phosphodiesterase inhibitor, a cRaf-MEK-ERF pathway activator, a p38 pathway activator, and mixtures thereof.

12. The method of claim 11 wherein the guanylyl cyclase stimulator is selected from the group consisting of atrial natriuretic peptide, C-type natriuretic peptide, 3-(5′-hydroxymethyl-2′-furyl)-1-benzylindazole, and mixtures thereof.

13. The method of claim 11 wherein the protein kinase G activator is selected from the group consisting of cGMP, 8-bromo-cGMP, 8-pCPT-cGMP, 8-dibutyl-cGMP, and mixtures thereof.

14. The method of claim 11 wherein the cGMP-specific phosphodiesterase inhibitor is selected from the group consisting of sildenafil, E4021, DMPPO, MY5441, zaprinast, and mixtures thereof.
